(19) United States
(12) Patent Application Publication
(10) Pub. No.: US 2007/0186298 A1
(43) Pub. Date: Aug. 9, 2007

(54) Follistatin gene as a genetic marker for reproductive and performance traits in pigs

(76) Inventor: Charlotte Dawn Blowe, Raleigh, NC (US)

Correspondence Address: Charlotte Dawn Blowe, 204 Essex Lane S., Clayton, NC, 27520, US

(21) Appl. No.: 11/324,526

(22) Filed: Jan. 4, 2006

(51) Int. Cl. A01K 67/027; C12Q 1/68

(52) U.S. Cl. 800/17; 435/6

(57) ABSTRACT

Disclosed herein are genetic markers for overall pig reproductive traits, including litter size, number born alive, number weaned, weaning weight, and number of stillborns, and performance traits, including days to reach 250 lbs., average daily gain, and feed to gain conversion, methods for identifying such markers, and methods of screening pigs to determine those more or less likely to possess these favorable reproductive and performance traits and more or less likely to produce litters with offspring who also have that same genetic ability to have these desirable traits and preferably selecting those pigs for future breeding purposes. The markers are based upon the presence or absence of certain polymorphisms in the pig follistatin gene.

### CROSS-REFERENCE TO RELATED APPLICATIONS

Current application claims priority of provisional patent No. 60/640,313. This application also claims priority of non provisional patent titled “Follistatin gene as a genetic marker for first parity litter size in pigs”, which was submitted on Dec. 30, 2005.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

Not applicable.

### BACKGROUND OF THE INVENTION

This invention relates generally to the detection of genetic differences for reproductive and performance traits among pigs and particularly use of a genetic marker in the follistatin gene, found on pig chromosome 16, which is indicative of the heritable traits of litter size, number born alive, number of fully formed fetuses, number of stillborns, number weaned, weaning weight, days to reach 250 lbs., average daily gain, and feed efficiency in pigs. There is also some indication that the invention may be of use for other reproductive and performance traits such as number of mummified fetuses and back fat.

There are numerous economically important traits in the swine production industry. These include growth and reproductive traits. In swine, there exists a store of genetic variation within and across breeds for these traits. This variation can be exploited by breeding techniques to attain animals with these desirable attributes. Growth traits have traditionally been easier to improve than reproductive traits. This is mostly because reproductive success is dependent upon a number of complex physiological events in both sexes. If genetic markers can be found which are linked to quantitative trait loci (QTL) affecting economic traits, especially reproductive traits in swine, these markers could be used to increase selection accuracy. It would be even more favorable if the marker associated with the reproductive trait was not also associated with a negative performance trait. It would be ideal if the marker associated with favorable reproductive traits was also associated with favorable growth traits.

The ability to pursue a specific beneficial genetic allele involves the identification of a DNA molecular indicator or marker for a major effect gene. The marker may be linked to a single gene with a major effect or linked to a quantity of genes with additive effects. DNA markers have several advantages; segregation is easily gauged and is explicit, and DNA markers are co-dominant, i.e., heterozygous and homozygous animals are easily distinguished. Another advantage is an increase in selection accuracy due to addition of information directly related to genotype. Still another advantage is the possibility of reducing generation interval by allowing selection to be made at an earlier age because this technique is not age or sex dependent. Once a marker system is ascertained selection evaluations could be made straightforwardly, since DNA markers can be analyzed any time after a genetic sample can be gathered from the individual infant animal.

The use of genetic disparity in genes has become a useful marker system for selection in reproduction. For example U.S. Pat. No. 5,374,526 issued Dec. 20, 1994 to Rothschild discloses a polymorphism in the pig estrogen receptor gene which is coupled with an increase in litter size, the disclosure of which is incorporated herein by reference. U.S. Pat. No. 5,935,784 issued on Aug. 10, 1999 to Rothschild et al., incorporated herein by reference, disclosed polymorphic markers in the pig prolactin receptor gene which are linked with larger litter size and general reproductive competence.

The current invention provides a genetic marker, established upon the finding of a polymorphism in the follistatin gene, which relates to reproductive and performance traits in pigs. This will permit genetic typing of pigs for their follistatin genes and for determination of the relationship of specific RFLPs to reproductive and growth traits. It will also permit the identification of individual males and females that carry the gene for improved reproductive and general performance traits. Thus, the markers may be selection tools in breeding programs to develop lines and breeds that produce litters containing offspring with more desirable characteristics.

According to the invention two polymorphisms previously identified in the follistatin gene in the pending patent titled “Follistatin gene as a marker for first parity litter size in pigs”, incorporated fully herein, have been found to be associated with several other reproductive traits including litter size for all parities, number born alive, number of fully formed fetuses, number of stillborns, number weaned and weaning weight. The polymorphisms are also correlated with growth traits such as days to 250 lbs., average daily gain and feed efficiency.

### BRIEF SUMMARY OF THE INVENTION

To achieve the objects and in agreement with the purpose of the invention, as embodied and generally described herein, the present invention provides a process for screening pigs to determine those which will be likely to perform reproductively and growth wise in an above average manner and offspring with these same above average performances when bred or to select against pigs which have alleles indicating the less favorable traits. Thus, the present invention provides a method for screening pigs to determine those more likely to have higher quality reproductive attributes and grow more efficiently themselves and offspring which will also possess the same qualities, and/or those less likely to perform below average, which method comprises the steps 1) obtaining a sample of genomic DNA from a pig; and 2) analyzing the genomic DNA obtained in 1) to determine which follistatin allele(s) is/are present. Briefly, a sample of genetic material is obtained from a pig, and the sample is analyzed to determine the presence or absence of at least one of the polymorphisms in the follistatin gene that is correlated with increased reproductive performance and more efficient growth.

In one embodiment the polymorphisms are restriction fragment length polymorphisms and the assay comprises identifying the pig follistatin gene from isolated pig genetic material; exposing the gene to a restriction endonuclease that yields restriction fragments of the gene of varying size; separating those restriction fragments to form a restriction pattern, such as by electrophoresis or HPLC separation; and contrasting the resulting restriction fragment pattern from a pig follistatin gene that is either known to have or not to have the desired marker. If a pig tests positive for the favorable marker, such pig can be considered for inclusion in the breeding program. If the pig does not test positive for the marker genotype the pig can be culled from the group and otherwise used.

In a most preferred embodiment the gene is isolated by the use of primers and DNA polymerase to amplify a specific region of the gene which contains one of the polymorphisms. Then the amplified region is digested with a restriction endonuclease and fragments are separated. Visualization of the RFLP pattern is by straightforward staining of the fragments, or by labeling the primers or the nucleoside triphosphates used in amplification.

It is also possible to ascertain linkage between precise alleles of other DNA markers and alleles of DNA markers known to be associated with a particular gene (e.g. the follistatin gene discussed herein), which have previously been found on the same chromosome. Thus, in the current condition, taking the follistatin gene, it would be possible, at least in the interim, to select for pigs expected to perform at a higher than average level reproductively and growth wise, or otherwise against pigs likely to perform below average, indirectly, by selecting for certain alleles of the follistatin associated marker through the selection of specific alleles of alternative chromosome 16 markers including S0111, S0006, S0077, S0390, S0326 and S0363.

The invention further comprises a kit for assessing a sample of pig DNA for the presence in pig genetic material of a preferred genetic marker located in the pig follistatin gene indicative of the heritable traits of litter size, number born alive, number of fully formed fetuses, number of stillborns, number weaned, weaning weight, days to reach 250 lbs., average daily gain and feed efficiency. At a minimum, the kit is a container with one or more reagents that identify a polymorphism either in or associated with the pig follistatin gene. Preferably, the reagent is a set of oligonucleotide primers capable of amplifying a fragment of the pig follistatin gene that contains one of the polymorphisms. The kit further may include a restriction enzyme that cleaves the pig follistatin gene in at least one place. In a most preferred embodiment the restriction enzyme is Mspl or Fnu4Hl or one which cuts at the same recognition sites.

The accompanying figures, which are incorporated herein and which constitute a part of this specification, illustrates one embodiment of the invention and, together with the description, serve to explain the principles of the invention.

It is an object of the invention to provide a method of screening pigs to determine those more likely to produce offspring containing these desirable traits. Another object of the invention is to provide a method for identifying genetic markers for pig reproductive and growth attributes.

A further object of the invention is to provide genetic markers for selection and breeding to obtain pigs that will be expected to have these desirable reproductive and development traits.

Yet another object of the invention is to provide a kit for evaluating a sample of pig DNA for specific genetic markers for reproductive and performance traits.

Supplementary objects and benefits of the invention will be set forth in part in the description that follows, and in part will be obvious from the description, or may be learned by the practice of the invention. The objects and advantages of the invention will be attained by means of the instrumentalities and amalgamations predominantly pointed out in the appended claims.

### DETAILED DESCRIPTION OF THE INVENTION

Reference will now be made in detail to the currently preferred quintessence of the invention, which together with the following examples, serve to explain the principles of the invention.

The invention relates to genetic markers associated with improved growth and reproductive performance in pigs. It provides a method of screening pigs to determine those more likely to produce larger, better quality litters and offspring with these favorable reproductive and growth traits when bred by identifying the presence or absence of a polymorphism in the follistatin gene that is correlated with the previously mentioned growth and reproductive traits. Used herein, the term “favorable reproductive traits” means a biologically significant increase in the following: litter size, number born alive, number of fully formed fetuses, number weaned above the mean of a given population and a decrease in the number of stillborns per litter. The term “favorable growth traits” or “favorable performance traits” means a decrease in the number of days to reach a weight of 250 lbs., a decrease in feed to gain ratio, and an increase in average daily gain and an increase in weaning weight when compared to the mean of a given population.

Thus, the invention correlates to genetic markers and techniques of recognizing those markers in a pig of a particular breed, strain, population, or group, whereby the selected pig is more likely to perform above average in the area of growth and reproduction for that particular breed, strain, population, or group. Any method of identifying the presence or absence of this marker may be used, including for example single-strand conformation polymorphism (SSCP) analysis, RFLP analysis, heteroduplex analysis, denaturing gradient gel electrophoresis, and temperature gradient electrophoresis, ligase chain reaction or even direct sequencing of the follistatin gene and examination for the Mspl or Fnu4Hl or other comparable enzymes' recognition pattern.

Other potential methods include non-gel systems such as TaqMan™. (Perkin Elmer). In this method oligonucleotide PCR primers are designed to border the mutation in question and allow amplification. A third oligonucleotide probe is then created to hybridize or bind to the region that contains the base found to be subject to change between different alleles of the follistatin gene. The probe is tagged with fluorescent dyes at both the 5′ and 3′ ends. The dyes are selected such that while in close proximity to each other the fluorescence of one of them is suppressed or quenched by the other and thus is undetectable. Extension carried out by Taq DNA polymerase from the 5′ primer leads to the excision of the attached dye at the 5′ end of the annealed probe as a result of the 5′ nuclease activity of the Taq DNA polymerase. This action removes the suppressing effect thus, allowing exposure of the fluorescence from the dye at the 3′ end of the probe. The discrimination between differing DNA sequences arises from the fact that if the binding of the probe to the template strand of DNA is incomplete, i.e. there is a mismatch of some sort, the cleavage of the dye does not occur. Thus only when the nucleotide sequence of the probe is entirely corresponding to the template molecule to which it is attached will quenching be eliminated. A reaction blend can contain two different probe sequences each one intended to detect different alleles that could be present thus allowing the exposure of both alleles in one reaction.

The use of RFLPs is the favored technique of detecting the polymorphisms. However, because the use of RFLP analysis relies ultimately on polymorphisms and DNA restriction sites along the nucleic acid molecule, other assays and techniques of detecting the polymorphisms can also be employed. Such procedures consist of ones that analyze the polymorphic gene product and identify polymorphisms by detecting the consequential distinctions in the gene product.

A RFLP analysis in general is a technique well-known to those skilled in the art. See, for example, U.S. Pat. No. 4,582,788 issued Apr. 15, 1986 to Erlich and U.S. Pat. No. 4,666,828 issued May 19, 1987 to Gusella, U.S. Pat. No. 4,772,549 issued Sep. 20, 1988 to Frossard, and U.S. Pat. No. 4,861,708 issued Aug. 29, 1989 to Frossard, all of which are incorporated herein by reference. Generally speaking, the technique requires obtaining the DNA to be studied, digesting the DNA with restriction endonucleases, separating the resulting fragments, and detecting the fragments of assorted genes.

In the present invention, a sample of genetic material is acquired from a pig. Samples can be obtained from blood, tissue, semen, hair, buccal swabs, saliva, etc. Generally, white blood cells are used as the source, and the genetic material is DNA. An adequate amount of cells are obtained to provide a sufficient amount of DNA for analysis. This amount will be known or readily determinable by those skilled in the art. The DNA is isolated from the blood cells or other suitable sample by techniques known to those skilled in the art.

Next the region containing the polymorphism is amplified by the use of primers and customary techniques, such as the polymerase chain reaction. This technique is described in U.S. Pat. No. 4,683,195, issued Jul. 28, 1987 to Mullis et al., U.S. Pat. No. 4,683,202, issued Jul. 28, 1987 to Mullis, U.S. Pat. No. 4,800,159 issued Jan. 24, 1989 to Mullis, et al., U.S. Pat. No. 4,889,818 issued Dec. 26, 1989 to Gelfand, et al., and U.S. Pat. No. 4,902,624, issued Feb. 20, 1990 to Clumbus, et al., all of which are incorporated herein by reference. The choice of primers is discussed in the references mentioned and incorporated herein. The primers should amplify the follistatin gene or at a minimum the portion of the follistatin gene which contains one of the polymorphic sites (or one associated with it) identified herein.

The amplified isolated DNA is then digested with a restriction endonuclease that cleaves or severs DNA hydrolytically at a restriction site which is a precise nucleotide sequence. These endonucleases, also referred to as restriction enzymes, are familiar to those trained in the art. For the present invention, a restriction enzyme should be chosen that cuts the pig follistatin gene in at least one location, creating at least a pair of gene fragments. An assessment is made as to whether or not any of the resulting fragments are polymorphic and if any particular polymorphism (RFLP) is linked with favorable reproductive or growth traits by methods known in the art in combination with the knowledge enclosed herein. Preferably, the restriction enzyme is Msp l for the FS1 marker or Fnu4Hl for the FS2 marker or other appropriate enzyme. The enzyme Msp l cleaves double stranded DNA at the sequence 5′ CCGG 3′, with the actual cut occurring between the pair of C's. The restriction enzyme Fnu4Hl cuts at the recognition sequence 5′ GCNGC 3′ with the cut occurring between the C and N. The quantity of enzyme to be added to the sample containing the pig DNA and the other suitable reagents for treating the sample will be unhesitatingly ascertainable to persons skilled in the art, given the information contained herein.

The resulting restriction fragments are then investigated by known practices that usually entail either the separation of fragments and visualization by staining or subsequent blotting and hybridization to acquire a particular pattern or the resolution of varying sizes of fragments. The preferred separation method is gel electrophoresis.

In this process, the digested DNA fragments are separated in a supporting medium based on size using an electric current. Gel sheets, such as agarose or similar substance, are normally used as the supporting medium. The sample, containing the restriction fragments, is added to the negative end of the gel. At least one molecular marker of known size is run on the same gel as a control to allow a size estimation of the restriction fragments. This technique usually permits a measure of resolution that separates DNA restriction fragments that differ in size from each other by as few as 100 base pairs.

In other embodiments, the resulting restriction fragments are denatured and relocated actually from the gel to a solid support, such as a nylon membrane, by contacting the gel with the filter in the existence of suitable reagents and under appropriate circumstances that encourage the transfer of the DNA from the gel to the membrane. These reagents and conditions are well-known to those skilled in the art. The relative positions of the fragments resulting from the separation process are sustained.

The next step entails the recognition of the assorted categories of sizes of the fragments or, otherwise, the detection of a particular sized fragment. The second may be of particular significance because it is a genetic marker that is correlated with favorable reproductive and growth traits. This is preferably achieved via staining the fragments with a chemical ethidium bromide.

Another procedure is the utilization of a hybridization probe. Such a probe is an oligonucleotide or polynucleotide that is adequately complementary or homologous to the fragments to bind with them, creating probe-fragment complexes. Preferably, the probe is a cDNA probe. The oligonucleotide or polynucleotide has a detectable entity attached to it that serves as a label. This allows the detection of the fragments, to which the probes are annealed. Probes are labeled by customary labeling methods, like a radiolabel, enzyme label, fluorescent label, biotin-avidin label, and the like. See U.S. Pat. No. 4,711,955 issued Dec. 8, 1987 to Ward et al. and U.S. Pat. No. 4,868,103 issued Sep. 19, 1989 to Stavrianopoulos et al., both of which are incorporated herein by reference.

The probes are placed in contact with the nylon membrane containing the restriction fragments for an ample length of time and under suitable hybridizing surroundings for the probes to attach to the fragments. The filter is then preferably rinsed to eliminate unattached probes and other unnecessary materials.

The probe-fragment complexes, which are hybridized to the filter, are then recognized by known methods. The fragments of interest are visualized according to the chosen detection method, which would be known to someone skilled in the art.

The recognition phase supplies a pattern, ensuing from the partitioning of the fragments based on size. Comparison of these fragments with molecular marker fragments of known size that have also been run on the same gel allows the approximation of size of the various fragments. The assorted polymorphisms in the pig follistatin gene are then determined by evaluation of the configurations created by similar assay of DNA from a number of different pigs. For some of the individual pigs, the patterns will vary from the normal pattern seen in most of the other pigs. This will be because of one or more restriction fragment length polymorphisms, i.e., restriction fragments of a varying length produced by the enzyme that cuts the pig follistatin gene. This illustrates different base pair sequences in such pigs.

Once a specific RFLP has been discovered, i.e., a restriction fragment of a particular length, a probe to this segment may be produced by the use of known methods. This allows different and quicker systems for detecting such polymorphism. For instance, once the DNA is digested, a sandwich hybridization format can be utilized. An assay of this type is disclosed in U.S. Pat. No. 4,486,539 issued Dec. 4, 1984 to Ranki, et al., and U.S. Pat. No. 4,563,419 issued Jan. 7, 1986 to Ranki, et al., both of which are incorporated herein by reference. The sample is brought into contact with a capture probe that is stationary on a solid carrier. The probe hybridizes the segment. Then the carrier is rinsed, and a labeled detection probe is added. After further rinsing, the detection probe is recognized, thus signifying the incidence of the preferred fragment.

In yet another embodiment, once the RFLP pattern has been determined or a specific polymorphic fragment has been discovered, it is compared to a second, fragment with a known RFLP pattern that is associated with favorable reproductive and growth traits. This second fragment has also been determined from the pig follistatin gene, using the same restriction endonuclease as the first and the same probe or an equal thereof under the same circumstances.

In a different embodiment of the invention, the restriction fragments can be identified by solution hybridization. In this process, the restriction fragments are bound with the probe and then separated. The separated probe-fragment complexes are then visualized as discussed above. Generally, they are seen on the gel without transport to filter paper.

In a most favored embodiment the polymorphism is detected by PCR amplification minus a probe. This technique is known to those of skill in the art and is disclosed in U.S. Pat. No. 4,795,699 entitled “DNA Polymerase” and U.S. Pat. No. 4,965,188. “Process for Amplifying, Detecting, and/or Cloning Nucleic Sequences Using a Thermostable Enzyme” both of which are incorporated herein by reference.

For this technique primers are designed to amplify the portion of the gene in which the polymorphism lies. Therefore, primers, which are preferably 4-30 bases, are constructed established on the sequence flanking the polymorphism including a forward 5′, primer and a reverse or anti-sense 3′ primer. The primers do not have to be the exact complement, and considerably equivalent sequences are also adequate. A DNA polymerase, such as Taq polymerase (many such polymerases are recognized and commercially accessible), is then added in the presence of the four nucleoside triphosphates and frequently a buffering agent. Detection is made possible by straightforward staining, such as with ethidium bromide, of separated PCR products to detect for products of predicted sizes based on the length of the amplified region. Reaction times, reagents, and primer design are all known to those skilled in the art and are discussed in the patents incorporated herein by reference. Additional PCR amplification may be combined with Single Strand Confirmation Polymorphism (SSCP).

Even though the previously mentioned techniques are depicted in terms of utilizing a solitary restriction enzyme and a lone set of primers, the methods are not so restricted. One or more supplementary enzymes and/or probes and/or primers can be used, if preferred. Additional enzymes, constructed probes and primers can be discovered through routine experimentation that could be performed by one skilled in the art.

Pig reproductive and growth genetic markers are determined as follows. Individuals from both sexes of the same background, i.e. breed, breed cross, or derived from similar genetic lineages and grown under regular conditions, are chosen. The reproductive and growth characteristics of each pig are determined. Restriction fragment length polymorphism analysis of the DNA is performed as discussed previously in order to determine polymorphisms in the follistatin gene of each pig. The polymorphisms are then related to each growth and reproductive trait of each individual animal. At least 20 and preferably a 100 pigs are used to make these determinations.

When this investigation is performed and the polymorphism(s) is/are determined by PCR-RFLP technique using the restriction endonuclease Mspl, Fnu4Hl or comparable enzyme and PCR primers may be created using corresponding known human follistatin sequences, or using pig follistatin gene sequence information as illustrated herein or even designed from sequences obtained using linkage data from nearby neighboring genes. According to the invention a primer set has been selected which amplifies a 625 bp fragment (forward primer 5′-GGACCGAGGAGGACGTAAAT-3′ (SEQ ID NO:1) and the reverse primer 5′-GGCCTTTCCAGGTGATGTTA-3′ (SEQ ID NO:2)). After PCR is performed, a restriction digest is carried out and restriction polymorphic fragments of approximately 125, 200, 225, and 425 base pairs are generated. The fragment of size 425 bp is designated as the favorable B allele, while the A allele is identified by the presence of two fragments of size 220 bp and 225 bp. The 125 base pair band is a monomorphic band. Another set of primers was also created for a second polymorphism closely related to the first one. These primers are 5′-TGCCGMTGAACAAGMGM-3′, SEQ ID NO:3, and 5′-CAGAAAACATCCCGACAGGT-3′, SEQ ID NO:4, which produces a product of 450 bp. Upon digestion of the amplified fragment with Fnu4Hl fragments of size 300, 200, 125, and 75 base pairs. The favorable B allele is identifiable by fragments of 125 bp and 75 bp in size, while the A allele is denoted by the presence of a 200 bp fragment. The 300 base pair fragment is a monomorphic fragment. The genotype associated with favorable reproductive and growth traits is BB for either set of primers and enzymes.

The reagents appropriate for employing the techniques of the invention may be packaged into convenient kits. The kits supply the essential materials, packaged into proper containers. At a bare minimum, the kit includes a reagent that recognizes a polymorphism in the pig follistatin gene that is associated with favorable reproductive and growth traits. Preferably, the reagent is a set of PCR reagents including a primer set, DNA polymerase, a suitable buffering agent, and 4 nucleoside triphosphates that bind with the pig follistatin gene or a portion thereof. Preferably, the PCR reagents and a restriction endonuclease that differentially cuts the pig follistatin gene in at least one place are included in the kit. In a predominantly preferred embodiment of the invention, the forward primer is SEQ ID NO:1 and the reverse primer is SEQ ID NO:2 and the restriction enzyme is Msp l, or the primer is SEQ ID NO:3 and 4 and the enzyme is Fnu4Hl. Preferably, the kit further includes additional materials, such as a sample collection vessel, DNA extracting reagents, reagents for detecting or visualizing the polymorphism, and a control. Additional reagents employed for hybridization, prehybridization, etc. may also be incorporated, if desired.

The techniques and resources of the invention may also be utilized in a more general manner to evaluate pig DNA, genetically type individual animals, and detect genetic differences existing among pigs. Specifically, a sample of pig genomic DNA may be assessed by reference to single or multiple controls to determine if a polymorphism in the follistatin gene is present. Preferably, PCR-RFLP analysis is executed with respect to the pig follistatin gene, and the outcomes are contrasted with a control. The control is the result of a PCR-RFLP analysis of the swine follistatin gene of a different individual where the polymorphism of the pig follistatin gene is known. Similarly, the follistatin genotype of a pig may be deciphered by acquiring a sample of its genomic material, performing PCR-RFLP analysis of the follistatin gene in the DNA, and evaluating the results with a control. Again, the control is the result of PCR-RFLP analysis of the follistatin gene of a different pig. The results genetically type the pig by indicating the polymorphism in its follistatin genes. Lastly, genetic differences among pigs can be discovered by attaining samples of the genomic DNA from at least a pair of pigs, recognizing the presence or absence of a polymorphism in the follistatin gene, and evaluating the results.

These techniques are functional for identifying the genetic markers relating to favorable reproductive and growth attributes, as discussed above, for distinguishing other polymorphisms in the follistatin gene that may be associated with other characteristics, and for the general scientific analysis of pig genotypes and phenotypes.

The genetic markers, protocols, and kits of the invention are also valuable in a breeding program to advance reproductive performance or growth in a breed, line, or population of pigs. Unremitting selection and propagation of sows that are at least heterozygous and preferably homozygous for a polymorphism associated with the favorable reproductive and growth would lead to a breed, line, or population being not just faster more efficient where growth is concerned, but more reproductively efficient as well. Hence, the markers are selection tools.

It is to be understood that the function of the teachings of the present invention to a definite dilemma or environment will be within the competence of one having ordinary skill in the art considering the information enclosed herein. The examples of the products and processes of the present invention emerge in the subsequent examples.

### EXAMPLE 1

Allele Segregation Verification: Because the populations tested in pending patent titled “Follistatin gene as a genetic marker for first parity litter size in pigs”, were small experimental populations, it was necessary to test a commercial population for the follistatin genetic markers.

Sample Collection: Genomic DNA can be extracted from any biological source as long as said source contains cell nuclei, for example white blood cells, sperm cells, hair follicles, buccal swabs, ear notches, skin, muscle, etc. The techniques, which follow, deal with white blood cells and tissue samples.

Buffy coat (white blood cells): Whole blood samples were collected from 165 purebred Chester White sows that had litter data for at least three parities. Samples were collected from each of the sows using vacutainers containing Tris EDTA. Blood samples were centrifuged at 4° C. for 20 minutes. The buffy coat was removed from each sample and placed into a labeled micro centrifuge tube. Blood samples were stored at −20° C.

DNA Extraction: There are various DNA extraction protocols available commercially and known to those skilled in the art. The techniques presented here are not the only method by which DNA can be extracted from a given sample type.

A mixture of two parts distilled water and one part buffy coat was put through the Gentra's protocol for the Generation DNA purification system extraction kit.

PCR Amplification: The method of PCR amplification is widely recognized and known to those skilled in the art. This process can vary in a multitude of factors, including, but not limited to, primers, enzymes, reaction conditions (times, temperatures, etc.) and reagents, all of which are intended to be included in this invention.

Primer design was described in detail in the pending patent titled “Follistatin gene as a genetic marker for first parity litter size in pigs”, which has been incorporated fully previously. The primers were: Follistatinl (FS1) forward: GGACCGAGGAGGACGTAAAT and reverse: GGCCTTTCCAGGTGATGTTA, Follistatin 2 (FS2) forward: TGCCGAATGMCAAGAAGAA and reverse: CAGAAAACATCCCGACAGGT

Other comparable primers were also designed for FS1 marker. They were: FSAF: AAACCGAACTGAGCAAGGAGGAGT, FSAR: ACTTCCAGTTCCGGCTGCTCTTTA, and FSBF: AAACCGAACTGAGCAAGGAGGAGT, FSBR: ATCTGGCCTTGAGGAGAGCACATT. The primers work at various annealing temperatures and under a multitude of reaction conditions and thermocycler profiles. One such setup is as follows: 2 micro liters DNA, 18 micro liters water, 2.5 micro liters 10× Taq Buffer, 1 micro liters DNTPs, 0.5 micro liters each of forward and reverse primer, 0.5 micro liters magnesium chloride, and 1 unit of Taq per reaction. The profile was:
a. 94° C. for 10 minutes
b. 94° C. for 30 sec
c. optimal annealing temperature (for FS1=56° C. , FS2=62° C.) for 30 sec
d. 72° C. for 130 sec
e. Go to step 2 for 35 cycles
f. 72° C. for 10 minutes
g. 4° C. forever
h. End

Each experiment described and its primers are but one incarnation and any primer consisting of at least 4 bases on each side of the Msp l or Fnu 4Hl polymorphic sites can be employed to amplify the intervening genetic sequence, which can then be subjected to the restriction enzymes listed or other such enzyme and examined for the presence of either of the markers. The primers, enzymes and methods disclosed are in no manner meant to limit this invention in the detection of markers in follistatin that show some form of correlation with the favorable growth and reproductive traits described in this patent.

Restriction Digest: Amplified PCR product from each individual sow was digested with either Msp l (FS1 any of the FS1 primer sets) or Fnu 4Hl (FS2) depending on the product. Manufacturer's temperature recommendations were followed for each enzyme. Twenty micro liter digests were conducted using 10 micro liters of PCR product, 7.7 micro liters of sterile water, 2 micro liters of buffer supplied with the enzyme, 0.2 micro liters BSA and 0.1 micro liters of enzyme. Digests were allowed to incubate at the enzyme's optimal temperature for three hours. Digest results were visualized using a 3% agarose gel with ethidium bromide. Digest results were run alongside uncut PCR product to ensure that the enzyme cut effectively.

Digest results were visualized on a 3% agarose gel with ethidium bromide to see if resulting banding patterns revealed a polymorphism.

Genotyping Chester White Sows for FS markers: Individuals were genotyped, and results were recorded. A couple of weeks later gel images were again scored to check for genotyping accuracy. Questionable genotypes were amplified again. Allele frequencies were calculated for the control and select lines.

The allele frequencies were as follows for the B allele: FS1=0.40 and FS2=0.40.

Arithmetic means were calculated for each reproductive trait recorded according to the genotype. Results are summarized according to parity and appear in Table 1.

TABLE 1
Genotypic Means for Each Reproductive Trait

| | AA | AB | BB |
|---|---|---|---|
| NBA1 | 8.9 | 8.7 | 9.9 |
| NSB1 | 1.2 | 1 | 0.6 |
| MUM1 | 0.1 | 0 | 0.1 |
| TNB1 | 10 | 9.7 | 11 |
| WW1 | 88 | 88 | 91 |
| TNW1 | 7.7 | 7.5 | 7.7 |
| NBA2 | 8.8 | 8.9 | 9.1 |
| NSB2 | 1.2 | 1 | 1.3 |
| MUM2 | 0.2 | 0.1 | 0.1 |
| TNB2 | 10 | 9.9 | 10 |
| WW2 | 95 | 99 | 90 |
| TNW2 | 7.5 | 7.7 | 7.3 |
| NBA3 | 9.4 | 9.6 | 10 |
| NSB3 | 1.1 | 1 | 1.1 |
| MUM3 | 0.1 | 0.1 | 0.1 |
| TNB3 | 11 | 11 | 11 |
| WW3 | 100 | 106 | 104 |
| TNW3 | 7.7 | 7.9 | 8.1 |
| NBA4 | 9.9 | 9.1 | 9.3 |
| NSB4 | 1.3 | 0.8 | 0.8 |
| MUM4 | 0.4 | 0.1 | 0.1 |
| TNB4 | 12 | 10 | 10 |
| WW4 | 92 | 97 | 94 |
| TNW4 | 7.7 | 7.5 | 6.5 |
| NBA5 | 8.2 | 8.7 | 9.1 |
| NSB5 | 2 | 0.8 | 1.7 |
| MUM5 | 0.3 | 0.2 | 0.7 |
| TNB5 | 11 | 9.7 | 11 |
| WW5 | 83 | 99 | 103 |
| TNW5 | 6.7 | 7.1 | 7.2 |
| NBA6 | 7.2 | 6.5 | 6.8 |
| NSB6 | 2.6 | 2.6 | 2.4 |
| MUM6 | 1.7 | 1.1 | 0.8 |
| TNB6 | 12 | 10 | 10 |
| WW6 | 63 | 59 | 22 |
| TNW6 | 5.1 | 4.7 | 2.2 |
| NBA7 | 6.8 | 8.2 | 4 |
| NSB7 | 1.5 | 1.1 | 3 |
| MUM7 | 0 | 0 | 0 |
| TNB7 | 8.3 | 9.3 | 7 |
| WW7 | 33 | 89 | 100 |
| TNW7 | 2 | 6.4 | 4 |

NBA = Number Born Alive,
NSB = Number of Stillborns,
MUM = Number of mummified fetuses,
TNB = Total Number Born,
WW = Weaning Weight,
TNW = total number weaned.
The numbers after the trait abbreviation represent the parity.

In over half the parities BB individuals performed the best when considering number born alive, number of mummified fetuses, and total number born.

Discussion and Conclusions: Because the alleles appear to be segregating and potentially useful in this population, more individuals were genotyped.

### EXAMPLE 2

Genotyping the remainder of the Chester White population used in example 1: The methods of example 1 were followed for the remainder Chester White population from the same example. However, because in all instances the FS2 test yielded the same results as the FS1 test, only FS1 was used. Ninety-eight additional Chester White sows were genotyped. This data was added to the data gathered in example 1. Allele frequencies were calculated for the entire Chester White population. They were: FS1-A allele=0.60, B allele=0.40.

A standard chi-square analysis was done to determine possible deviations from Hardy-Weinberg equilibrium. The marker was not expected to be in Hardy-Weinberg equilibrium due to violation of the assumption of no selection. In the swine industry there is a lot of selection pressure placed on populations in order to increase the occurrence of desirable traits. This is especially true when dealing with a purebred, breedstock herd, as is the case here. Deviations from an ideal population in Hardy-Weinberg equilibrium were significant for the marker in this population (P value 1.43×10⁻⁵).

Additive (a) and dominance (d) effects were calculated using estimate statements with orthogonal contrasts of solutions for genotypic effects. Additive effect is defined as half the difference between LS means of the two homozygotes. Dominance effect is the heterozygote LS mean minus the average of the two homozygote LS means. Additive and dominance effects are listed in Tables 2 and 3.

TABLE 2
Additive effects of B allele for FS1 marker

| Trait | Effect | Standard Error | P value |
|---|---|---|---|
| # Born Alive | 0.545 | 0.297 | 0.067 |
| Birth Weight | 0.084 | 1.05 | 0.42 |
| # Stillborn | −0.32 | 0.202 | 0.114 |
| Mummies | −0.09 | 0.11 | 0.4 |
| Weaning Wt | 3.8 | 4.2 | 0.36 |
| # Weaned | 0.088 | 0.31 | 0.78 |

TABLE 3
Dominance effects of B allele for FS1 marker

| Trait | Effect | Standard Error | P value |
|---|---|---|---|
| # Born Alive | −0.524 | 0.18 | 0.037 |
| Birth Weight | −1.25 | 0.63 | 0.49 |
| # Stillborn | −0.154 | 0.122 | 0.208 |
| Mummies | −0.05 | 0.06 | 0.45 |
| Weaning Wt | −0.21 | 2.5 | 0.93 |
| # Weaned | −0.14 | 0.19 | 0.45 |

Data were analyzed using PROC GLM (SAS Inst., Inc., Cary, N.C.). The model included fixed effects of genotype and parity. Dependent variables were number born alive, litter birth weight, number of stillborns, number of mummified fetuses, litter weaning weight, and number weaned. Genotype was found to be significant when considering number born alive and number of stillborns (P value=0.0085 and P value=0.0309, respectively).

Least square (LS) means were calculated for each trait and are presented in Table 4. When looking at pair wise differences of LS means for each genotype when considering number born alive (NBA), BB differed significantly from AA and AB (P=0.067, 0.004, respectively). When examining birth weight (BW) AB tended to differ significantly from BB (P=0.088). In the case of number of stillborns (NSB) AA differed significantly from AB (P=0.01).

TABLE 4
Least Square Means for Litter Traits and Standard Errors.

| | NBA | SE | BW | SE | NSB | SE | MUM | SE | WW | SE | NW | SE |
|---|---|---|---|---|---|---|---|---|---|---|---|---|
| AA | 8.8 | 0.15 | 30.5 | 0.53 | 1.4 | 0.1 | 0.3 | 0.05 | 90.6 | 2.1 | 7.1 | 0.16 |
| AB | 8.6 | 0.11 | 29.7 | 0.37 | 1.1 | 0.07 | 0.21 | 0.04 | 92.3 | 1.5 | 7 | 0.11 |
| BB | 9.4 | 0.26 | 31.4 | 0.92 | 1.1 | 0.18 | 0.21 | 0.93 | 94.4 | 3.7 | 7.2 | 0.27 |

Estimated breeding values (EBV) for several traits were also examined for trends related to the follistatin marker. The traits were: litter size (EBVLS), number weaned (EBVNW), litter weight (EBVLW), days to 250 lbs. (EBVDAYS), backfat (EBVBF), loin eye area (EBVLEA), average daily gain (EBVADG), feed to gain ratio (EBVFG), average depth of fat (EBVADF). Additive and dominance effects were calculated for each. The results appear in Tables 5 and 6.

TABLE 5
Additive effects of B allele for FS1 marker

| Trait | Effect | Standard Error | P value |
|---|---|---|---|
| EBVLS | 0.11 | 0.06 | 0.085 |
| EBVNW | −0.005 | 0.018 | 0.78 |
| EBVLW | −0.02 | 0.68 | 0.98 |
| EBVDAYS | −1.67 | 0.76 | 0.029 |
| EBVBF | −0.017 | 0.018 | 0.35 |
| EBVLEA | −0.062 | 0.034 | 0.07 |
| EBVADG | 0.039 | 0.017 | 0.022 |
| EBVFG | −.035 | 0.015 | 0.202 |
| EBVADF | 0.0026 | 0.015 | 0.86 |

TABLE 6
Dominance effects of B allele for FS1 marker

| Trait | Effect | Standard Error | P value |
|---|---|---|---|
| EBVLS | −0.07 | 0.04 | 0.08 |
| EBVNW | 0.012 | 0.01 | 0.28 |
| EBVLW | −0.07 | 0.43 | 0.87 |
| EBVDAYS | 0.17 | 0.48 | 0.72 |
| EBVBF | −0.0003 | 0.01 | 0.98 |
| EBVLEA | 0.049 | 0.02 | 0.021 |
| EBVADG | −0.004 | 0.011 | 0.67 |
| EBVFG | 0.0026 | 0.009 | 0.78 |
| EBVADF | −0.001 | 0.01 | 0.98 |

The EBV data were analyzed using PROC GLM (SAS Inst., Inc., Cary, N.C.). The model included fixed effect of genotype. Dependent variables were litter size (EBVLS), number weaned (EBVNW), litter weight (EBVLW), days to 250 lbs. (EBVDAYS), backfat (EBVBF), loin eye area (EBVLEA), average daily gain (EBVADG), feed to gain ratio (EBVFG), average depth of fat (EBVADF). Genotype was found to be significant when considering the breeding value for loin eye area (P=0.04). Genotype tended to be significant when considering the breeding values for litter size, days to 250 lbs., average daily gain, and feed to gain ratio (P=0.10, 0.09, 0.067, 0.059, respectively).

Least square (LS) means were calculated for each trait and are presented in Table 7. When looking at pair wise differences of LS means for each genotype when considering litter size EBV, BB tended to differ from AA and differed significantly from AB (P=0.085, 0.033, respectively). When examining EBV for days to reach 250 lbs., BB differed significantly from AA (P=0.029), In the case of loin eye area EBV, BB differed significantly from both AA and AB (P=0.07, 0.012, respectively). In the instance of average daily gain breeding values BB differed significantly from AA (P=0.022). The BB genotype also differed significantly from AA when considering feed to gain ratio EBV (P=0.02).

TABLE 7
a. Least Square Means for reproductive traits Estimated
Breeding Values

| | LS | SE | NW | SE | LW | SE |
|---|---|---|---|---|---|---|
| AA | 0 | .04 | .002 | .01 | −.3 | .4 |
| AB | −.1 | .02 | .01 | .01 | −.4 | .3 |
| BB | .08 | .05 | 0 | .02 | −.3 | .6 |

b. Least Square Means for growth traits Estimated
Breeding Values

| | DAYS | SE | BF | SE | LEA | SE | ADG | SE | FG | SE | ADF | SE |
|---|---|---|---|---|---|---|---|---|---|---|---|---|
| AA | −.5 | .4 | .04 | .01 | −.05 | .02 | .01 | .01 | .01 | .01 | .03 | .01 |
| AB | −1.2 | .3 | .03 | .07 | −.03 | .01 | .02 | .01 | 0 | .01 | .03 | .01 |
| BB | −2.2 | .6 | .02 | .02 | −.11 | .03 | .05 | .01 | 0 | .01 | .03 | .01 |

Discussion and Conclusions: A candidate gene approach has been engaged to locate a major gene for economically important traits in the livestock industry. Rothschild et al. proposed the estrogen receptor gene as a gene associated with a major gene of litter size (Rothschild et al., P.N.A.S. USA, 93:201-205 (1996)), incorporated herein by reference. The present inventor has examined the follistatin gene as a candidate gene to investigate its effect on pig reproductive and growth traits. Significant additive effects were found for NBA when considering phenotypic and breeding value data. This implies that adding another copy of the B allele to an animal increases the NBA per litter. Significant additive effects were also found for breeding value data when considering days to reach 250 lbs. and average daily gain. This implies that BB individuals are genetically more likely to reach the economically important weight of 250 lbs. and be more feed efficient in the process. The least squared means for phenotypic data were all in the favorable direction when comparing BB individuals to the alternative genotypes. The favorable genotype had larger numbers of pigs born alive per litter, weaned more and heavier pigs per litter, and had fewer stillborns and mummies than the alternate genotypes. These results indicate that there is a tendency for the follistatin markers to be associated with reproductive and growth traits in purebred Chester White animals.

### EXAMPLE 3

Genotyping a second population of Chester Whites for Follistatin Marker: Because of the implications of the statistics a second population of purebred Chester Whites was also genotyped for the follistatin marker to see if the trends would hold true for another population.

The methods of example 1 were followed for the second Chester White population. Again, only FS1 was used for genotyping. Seventy-seven Chester White sows were genotyped. Allele frequencies were calculated for the entire Chester White population. They were: FS1-A allele=0.69,B allele=0.31.

A standard chi-square analysis was done to determine possible deviations from Hardy-Weinberg equilibrium. The marker was not expected to be in Hardy-Weinberg equilibrium due to violation of the assumption of no selection and no random genetic drift. In the swine industry there is a lot of selection pressure placed on populations in order to increase the occurrence of desirable traits. This is especially true when dealing with a purebred, breedstock herd, as is the case here. Also this population of sows was small, so random genetic drift should be occurring in this population. The population did not deviate from an ideal population when examining FS1 allele frequencies.

Additive (a) and dominance (d) effects were calculated using estimate statements with orthogonal contrasts of solutions for genotypic effects. Additive effect is defined as half the difference between LS means of the two homozygotes. Dominance effect is the heterozygote LS mean minus the average of the two homozygote LS means. Additive and dominance effects are listed in Tables 8 and 9. None of these effects were statistically significant. With such a small sample size, it is difficult to get significant statistics.

TABLE 8
Additive effects of B allele for FS1 marker

| Trait | Effect | Standard Error | P value |
|---|---|---|---|
| # Born Alive | −1.53 | 1.27 | 0.23 |
| Birth Weight | −0.19 | 3.9 | 0.96 |
| # Stillborn | −0.12 | 0.63 | 0.85 |
| Mummies | −0.08 | 0.32 | 0.79 |
| Weaning Wt | 6 | 22.6 | 0.79 |
| # Weaned | 0.34 | 1.34 | 0.8 |

TABLE 9
Dominance effects of B allele for FS1 marker

| Trait | Effect | Standard Error | P value |
|---|---|---|---|
| # Born Alive | −0.88 | 0.77 | 0.25 |
| Birth Weight | −3 | 2.3 | 0.2 |
| # Stillborn | −0.09 | 0.38 | 0.81 |
| Mummies | 0.09 | 0.19 | 0.63 |
| Weaning Wt | 6.9 | 13.7 | 0.62 |
| # Weaned | 0.29 | 0.81 | 0.72 |

Data were analyzed using PROC GLM (SAS Inst., Inc., Cary, N.C.). The model included fixed effects of genotype and parity. Dependent variables were number born alive, litter birth weight, number of stillborns, number of mummified fetuses, litter weaning weight, and number weaned. Genotype was found to be significant when considering number born alive (P value=0.0259).

Least square (LS) means were calculated for each trait and are presented in Table 10. When looking at pair wise differences of LS means for each genotype when considering number born alive (NBA), AA differed significantly from AB (P=0.0097). When examining birth weight (BW) AB tended to differ significantly from AA (P=0.10).

TABLE 10
Least Square Means for Litter Traits and Standard Errors.

| | NBA | SE | BW | SE | NSB | SE | MUM | SE | WW | SE | NW | SE |
|---|---|---|---|---|---|---|---|---|---|---|---|---|
| AA | 10.7 | 0.46 | 30.9 | 1.4 | 0.98 | 0.23 | 0.21 | 0.12 | 107 | 8.3 | 7.3 | 0.49 |
| AB | 9.1 | 0.5 | 27.8 | 1.5 | 0.83 | 0.24 | 0.26 | 0.13 | 117 | 8.9 | 7.8 | 0.53 |
| BB | 9.2 | 1.2 | 30.7 | 3.7 | 0.86 | 0.61 | 0.13 | 0.31 | 113 | 21.7 | 7.6 | 0.28 |

The same statistics were also run using estimated breeding values for the following traits: litter size (EBVLS), number weaned (EBVNW), litter weight (EBVLW), days to 250 lbs. (EBVDAYS), backfat (EBVBF), loin eye area (EBVLEA), average daily gain (EBVADG), feed to gain ratio (EBVFG), average depth of fat (EBVADF). Additive and dominance effects are summarized in tables 11 and 12.

TABLE 11
Additive effects of B allele for FS1 marker

| Trait | Effect | Standard Error | P value |
|---|---|---|---|
| EBVLS | −0.22 | 0.16 | 0.16 |
| EBVNW | 0.06 | 0.03 | 0.1 |
| EBVLW | 1.97 | 1.4 | 0.16 |
| EBVDAYS | 0.54 | 1.5 | 0.71 |
| EBVBF | −0.04 | 0.03 | 0.17 |
| EBVLEA | 0.07 | 0.08 | 0.37 |
| EBVADG | −0.01 | 0.03 | 0.76 |
| EBVFG | −0.015 | 0.03 | 0.57 |
| EBVADF | −0.03 | 0.03 | 0.2 |

TABLE 12
Dominance effects of B allele for FS1 marker

| Trait | Effect | Standard Error | P value |
|---|---|---|---|
| EBVLS | −0.05 | 0.11 | 0.68 |
| EBVNW | 0.03 | 0.02 | 0.29 |
| EBVLW | 0.36 | 0.97 | 0.71 |
| EBVDAYS | 1.1 | 1.03 | 0.30 |
| EBVBF | 0.004 | 0.02 | 0.84 |
| EBVLEA | −0.01 | 0.05 | 0.89 |
| EBVADG | −0.02 | 0.02 | 0.3 |
| EBVFG | 0.02 | 0.02 | 0.32 |
| EBVADF | −0.01 | 0.02 | 0.68 |

Least square (LS) means were calculated for each trait and are presented in Table 13. When looking at pair wise differences of LS means for each genotype when considering number weaned EBV, AA tended to differ from BB and differed significantly from AB (P=0.1, 0.02, respectively).

TABLE 13
a. Least Square Means for Reproductive Trait Estimated
Breeding Values

| | LS | SE | NW | SE | LW | SE |
|---|---|---|---|---|---|---|
| AA | .25 | .07 | 0 | .02 | −.21 | .6 |
| AB | .09 | .08 | .04 | .02 | 1.1 | .7 |
| BB | .03 | .14 | .04 | .03 | 1.8 | 1.2 |

b. Least Square Means for Growth Trait Estimated
Breeding Values

| | DAYS | SE | BF | SE | LEA | SE | ADG | SE | FG | SE | ADF | SE |
|---|---|---|---|---|---|---|---|---|---|---|---|---|
| AA | −2.2 | .64 | 0 | .01 | −.02 | .03 | .05 | .01 | −.03 | .01 | .02 | .01 |
| AB | −.87 | .72 | −.02 | .01 | .01 | .04 | .02 | .02 | −.02 | .01 | −.01 | .01 |
| BB | −1.7 | 1.3 | −.04 | .03 | .05 | .07 | .04 | .03 | −.05 | .02 | −.01 | .02 |

Discussion and Conclusions: The only significant additive effect was for breeding value of number weaned. The LS means were in the favorable direction for BB individuals for the breeding value traits of litter weight, backfat and feed to gain ratio. While these results do not confirm the findings of examples 1 and 2, it is necessary to remember that this population had a very small number of BB individuals (only 9). It is possible that given a larger sample size, the results would be similar to examples 1 and 2.

### EXAMPLE 4

Genotyping a third population of purebred Chester Whites: Because of the implications of the statistics from examples 1 and 2 and the lack of significant findings in example 3, a third population of purebred Chester Whites was also genotyped for the follistatin marker to see which way the trends would go.

The methods of example 1 were followed for the third Chester White population. Again, only FS1 was used for genotyping. One hundred and twenty-nine Chester White sows were genotyped. Allele frequencies were calculated for the entire Chester White population. They were: FS1-A allele=0.62, B allele =0.38.

A standard chi-square analysis was done to determine possible deviations from Hardy-Weinberg equilibrium. The marker was not expected to be in Hardy-Weinberg equilibrium due to violation of the assumption of no selection and no random genetic drift. In the swine industry there is a lot of selection pressure placed on populations in order to increase the occurrence of desirable traits. This is especially true when dealing with a purebred, breedstock herd, as is the case here. Also this population of sows was small, so random genetic drift should be occurring in this population. The population did not deviate from an ideal population when examining FS1 allele frequencies.

Additive (a) and dominance (d) effects were calculated using estimate statements with orthogonal contrasts of solutions for genotypic effects. Additive effect is defined as half the difference between LS means of the two homozygotes. Dominance effect is the heterozygote LS mean minus the average of the two homozygote LS means. Additive and dominance effects are listed in Tables 14 and 15. None of these effects were statistically significant. With such a small sample size, it is difficult to get significant statistics.

TABLE 14
Additive effects of B allele for FS1 marker

| Trait | Effect | Standard Error | P value |
|---|---|---|---|
| # Born Alive | 0.35 | 0.77 | 0.65 |
| Birth Weight | 0.53 | 1.7 | 0.76 |
| # Stillborn | 0.06 | 0.41 | 0.89 |
| Mummies | −0.08 | 0.1 | 0.43 |
| Weaning Wt | 7.7 | 8.9 | 0.39 |
| # Weaned | 0.35 | 0.77 | 0.65 |

TABLE 15
Dominance effects of B allele for FS1 marker

| Trait | Effect | Standard Error | P value |
|---|---|---|---|
| # Born Alive | 0.46 | 0.54 | 0.4 |
| Birth Weight | 0.92 | 1.2 | 0.44 |
| # Stillborn | −0.27 | 0.29 | 0.34 |
| Mummies | −0.02 | 0.07 | 0.72 |
| Weaning Wt | −0.44 | 6.2 | 0.9 |
| # Weaned | 0.49 | 0.46 | 0.29 |

Data were analyzed using PROC GLM (SAS Inst., Inc., Cary, N.C.). The model included fixed effects of genotype and parity. Dependent variables were number born alive, litter birth weight, number of stillborns, number of mummified fetuses, litter weaning weight, and number weaned. Genotype was not found to be significant for any of the traits.

Least square (LS) means were calculated for each trait and are presented in Table 16. When looking at pair wise differences of LS means for each genotype when considering number weaned (NW), AA differed significantly from AB (P=0.01).

TABLE 16
Least Square Means for Litter Traits and Standard Errors.

| | NBA | SE | BW | SE | NSB | SE | MUM | SE | WW | SE | NW | SE |
|---|---|---|---|---|---|---|---|---|---|---|---|---|
| AA | 9.04 | 0.88 | 18.1 | 1.9 | 0.62 | 0.47 | 0.04 | 0.11 | 111.5 | 10.2 | 7.9 | 0.75 |
| AB | 9.7 | 0.92 | 19.3 | 2.03 | 0.38 | 0.49 | −0.02 | 0.12 | 114.9 | 10.6 | 8.7 | 0.78 |
| BB | 9.4 | 1.03 | 18.6 | 2.3 | 0.68 | 0.55 | −0.03 | 0.13 | 119.1 | 11.9 | 8.5 | 0.87 |

The same statistics were also run using estimated breeding values for the following traits: litter size (EBVLS), number weaned (EBVNW), litter weight (EBVLW), days to 250 lbs. (EBVDAYS), backfat (EBVBF), loin eye area (EBVLEA), average daily gain (EBVADG), feed to gain ratio (EBVFG), average depth of fat (EBVADF). Genotype was found to be significant when considering the breeding value for number weaned and litter weight (P=0.0017, 0.0315, respectively). Additive and dominance effects are summarized in tables 17 and 18.

TABLE 17
Additive effects of B allele for FS1 marker

| Trait | Effect | Standard Error | P value |
|---|---|---|---|
| EBVLS | −0.05 | 0.08 | 0.51 |
| EBVNW | 0.07 | 0.02 | 0.0004 |
| EBVLW | 1.7 | 0.63 | 0.009 |
| EBVDAYS | −0.12 | 0.81 | 0.88 |
| EBVBF | 0.01 | 0.01 | 0.5 |
| EBVLEA | 0.007 | 0.03 | 0.82 |
| EBVADG | 0.002 | 0.02 | 0.92 |
| EBVFG | 0.004 | 0.15 | 0.78 |
| EBVADF | 0.009 | 0.01 | 0.52 |

TABLE 18
Dominance effects of B allele for FS1 marker

| Trait | Effect | Standard Error | P value |
|---|---|---|---|
| EBVLS | −0.04 | 0.05 | 0.47 |
| EBVNW | −0.01 | 0.014 | 0.45 |
| EBVLW | −0.56 | 0.43 | 0.2 |
| EBVDAYS | 0.92 | 0.56 | 0.1 |
| EBVBF | −0.01 | 0.01 | 0.33 |
| EBVLEA | 0.01 | 0.02 | 0.58 |
| EBVADG | −0.02 | 0.01 | 0.11 |
| EBVFG | 0.008 | 0.01 | 0.43 |
| EBVADF | −0.02 | 0.01 | 0.08 |

Least square (LS) means were calculated for each trait and are presented in Table 19. When looking at pair wise differences of LS means for each genotype when considering number weaned EBV, BB tended to differ from AB and differed significantly from AA (P=0.064, 0.0004, respectively). When examining litter weight EBV BB differed significantly from AA (P=0.0091).

TABLE 19
a. Least Square Means for Reproductive Trait Estimated Breeding Values

| | LS | SE | NW | SE | LW | SE |
|---|---|---|---|---|---|---|
| AA | .06 | .04 | −.02 | .01 | −.28 | .31 |
| AB | 0 | .04 | .01 | .01 | −.01 | .3 |
| BB | .01 | .07 | .05 | .02 | 1.4 | .55 |

b. Least Square Means for Growth Trait Estimated Breeding Values

| | DAYS | SE | BF | SE | LEA | SE | ADG | SE | FG | SE | ADF | SE |
|---|---|---|---|---|---|---|---|---|---|---|---|---|
| AA | −.96 | .4 | −.01 | .01 | −.03 | .02 | .02 | .01 | −.02 | .01 | .003 | .01 |
| AB | −.1 | .4 | −.01 | .01 | −.01 | .02 | .003 | .01 | −.01 | .01 | −.01 | .01 |
| BB | −1.1 | .7 | 0 | .01 | −.02 | .03 | .02 | .02 | −.02 | .01 | .01 | .01 |

Discussion and Conclusions: The data from this population showed significant additive effects for breeding values of number weaned and litter weight, indicating an advantage for each additional B allele when considering breeding values for number weaned and litter weight. A significant dominance effect was also found for breeding value for days to 250 lbs signifying an advantage for heterozygous individuals for this trait. The least square means for the BB genotype were in the favorable direction for phenotypic traits mummies and weaning weight, and breeding values for number weaned, litter weight and days to 250 lbs. These results are not a replica of examples 1 and 2, but they do show that the follistatin markers may be useful in selecting for growth and reproductive traits.

### EXAMPLE 5

Allele Segregation in Purebred Yorkshires and Purebred Landrace Animals: For a genetic marker to be the most useful, it must be informative in as many breeds as possible. Sixty-four purebred Yorkshire and twenty-one Landrace sows were tested for the FS1 marker following the methods of example 1. Allele frequencies were calculated for the each breed population.

| | | |
|---|---|---|
| Yorkshire | A allele = 0.74 | B allele = 0.26 |
| Landrace | A allele = 0.71 | B allele = 0.29 |

Even though the sample sizes for these two breeds are very small, especially in the Landrace population, the same statistics were run, just to see if any trends emerged early from the data.

A standard chi-square analysis was done to determine possible deviations from Hardy-Weinberg equilibrium. The marker was not expected to be in Hardy-Weinberg equilibrium due to violation of the assumption of no selection and no random genetic drift. In the swine industry there is a lot of selection pressure placed on populations in order to increase the occurrence of desirable traits. This is especially true when dealing with a purebred, breedstock herd, as is the case here. Also this population of sows was small, so random genetic drift should be occurring in this population. The Yorkshire population deviated from an ideal population when examining FS1 allele frequencies (P=0.01) while the Landrace group did not deviate.

Additive (a) and dominance (d) effects were calculated using estimate statements with orthogonal contrasts of solutions for genotypic effects. Additive effect is defined as half the difference between LS means of the two homozygotes. Dominance effect is the heterozygote LS mean minus the average of the two homozygote LS means. Additive and dominance effects are listed in Tables 20, 21, 22 and 23. None of these effects were statistically significant. With such a small sample size, it is difficult to get significant statistics.

TABLE 20
Additive effects of B allele for FS1 marker for Yorkshires

| Trait | Effect | Standard Error | P value |
|---|---|---|---|
| # Born Alive | 1.14 | 1.2 | 0.35 |
| Birth Weight | 0.79 | 2.54 | 0.76 |
| # Stillborn | −0.6 | 0.36 | 0.1 |
| Mummies | 0 | 0 | — |
| Weaning Wt | 11 | 10.6 | 0.3 |
| # Weaned | 1.0 | 0.91 | 0.27 |

TABLE 21
Dominance effects of B allele for FS1 marker for Yorkshires

| Trait | Effect | Standard Error | P value |
|---|---|---|---|
| # Born Alive | −0.09 | 0.95 | 0.93 |
| Birth Weight | −1.14 | 2.01 | 0.57 |
| # Stillborn | 10.21 | 0.29 | 0.46 |
| Mummies | 0 | 0 | — |
| Weaning Wt | −11.4 | 8.40 | 0.18 |
| # Weaned | −0.79 | 0.72 | 0.28 |

TABLE 22
Additive effects of B allele for FS1 marker for Landrace

| Trait | Effect | Standard Error | P value |
|---|---|---|---|
| # Born Alive | 0.5 | 2.98 | 0.87 |
| Birth Weight | 2.6 | 6 | 0.67 |
| # Stillborn | −0.3 | 0.62 | 0.63 |
| Mummies | 0 | 0 | — |
| Weaning Wt | 9.4 | 30.7 | 0.76 |
| # Weaned | 0.6 | 2.6 | 0.82 |

TABLE 23
Dominance effects of B allele for FS1 marker for Landrace

| Trait | Effect | Standard Error | P value |
|---|---|---|---|
| # Born Alive | −2.7 | 1.7 | 0.15 |
| Birth Weight | −5.9 | 3.5 | 0.11 |
| # Stillborn | 0.15 | 0.36 | 0.68 |
| Mummies | 0 | 0 | — |
| Weaning Wt | 3.9 | 17.9 | 0.83 |
| # Weaned | 0.3 | 1.5 | 0.85 |

Data were analyzed using PROC GLM (SAS Inst., Inc., Cary, N.C.) for each breed. The model included fixed effects of genotype and parity. Dependent variables were number born alive, litter birth weight, number of stillborns, number of mummified fetuses, litter weaning weight, and number weaned. Genotype was not found to be significant for any of the traits in either breed's data.

Least square (LS) means were calculated for each trait and are presented in Tables 24 and 25. When looking at pair wise differences of LS means for each genotype in the Yorkshire when considering number of stillborns (NSB), AA tended to differ significantly from BB (P=0.1) and differed from AB (P=0.06). For the Landrace data, genotype AA tended to differ significantly from AB for both NBA and BW (P=0.08, 0.09 respectively).

TABLE 24
Least Square Means for Litter Traits and Standard Errors for Yorkshires

| | NBA | SE | BW | SE | NSB | SE | MUM | SE | WW | SE | NW | SE |
|---|---|---|---|---|---|---|---|---|---|---|---|---|
| AA | 9.7 | 1.07 | 20.1 | 2.3 | 1.65 | 0.32 | 0 | 0 | 125 | 9.5 | 8.9 | 0.81 |
| AB | 10.2 | 0.98 | 19.3 | 2.1 | 1.1 | 0.3 | 0 | 0 | 119 | 8.7 | 8.6 | 0.74 |
| BB | 10.9 | 1.3 | 20.9 | 2.7 | 1.1 | 0.39 | 0 | 0 | 136 | 11.3 | 9.9 | 0.97 |

TABLE 25
Least Square Means for Litter Traits and Standard Errors for Landrace

| | NBA | SE | BW | SE | NSB | SE | MUM | SE | WW | SE | NW | SE |
|---|---|---|---|---|---|---|---|---|---|---|---|---|
| AA | 10.5 | 0.9 | 21.4 | 1.8 | 0.3 | 0.19 | 0 | 0 | 101 | 9.3 | 8.4 | 0.8 |
| AB | 8.1 | 0.9 | 16.8 | 1.8 | 0.3 | 0.19 | 0 | 0 | 109 | 9.3 | 9 | 0.8 |
| BB | 11 | 0.8 | 24 | 5.7 | 0 | 0.59 | 0 | 0 | 110 | 29.3 | 9 | 2.5 |

The same statistics were also run using estimated breeding values for the following traits: litter size (EBVLS), number weaned (EBVNW), litter weight (EBVLW), days to 250 lbs. (EBVDAYS), backfat (EBVBF), loin eye area (EBVLEA), average daily gain (EBVADG), feed to gain ratio (EBVFG), average depth of fat (EBVADF). Genotype was found to be significant when considering the breeding value for litter size for the Yorkshire group (P=0.016). Additive and dominance effects are summarized in tables 26, 27, 28 and 29.

TABLE 26
Additive effects of B allele for FS1 marker for Yorkshires

| Trait | Effect | Standard Error | P value |
|---|---|---|---|
| EBVLS | 0.14 | 0.066 | 0.036 |
| EBVNW | 0.02 | 0.025 | 0.39 |
| EBVLW | 0.72 | 0.88 | 0.41 |
| EBVDAYS | −0.54 | 1.1 | 0.64 |
| EBVBF | −0.01 | 0.02 | 0.57 |
| EBVLEA | −0.02 | 0.045 | 0.55 |
| EBVADG | −0.013 | 0.025 | 0.60 |
| EBVFG | −0.015 | 0.019 | 0.42 |
| EBVADF | −0.004 | 0.021 | 0.85 |

TABLE 27
Additive effects of B allele for FS1 marker for Landrace

| Trait | Effect | Standard Error | P value |
|---|---|---|---|
| EBVLS | −0.05 | 0.25 | 0.83 |
| EBVNW | −0.1 | 0.06 | 0.1 |
| EBVLW | −1.9 | 1.9 | 0.32 |
| EBVDAYS | 1.8 | 3.3 | 0.59 |
| EBVBF | −0.02 | 0.08 | 0.8 |
| EBVLEA | −0.07 | 0.15 | 0.66 |
| EBVADG | −0.04 | 0.07 | 0.6 |
| EBVFG | 0.02 | 0.05 | 0.76 |
| EBVADF | −0.03 | 0.07 | 0.68 |

TABLE 28
Dominance effects of B allele for FS1 marker Yorkshires

| Trait | Effect | Standard Error | P value |
|---|---|---|---|
| EBVLS | 0.06 | 0.056 | 0.28 |
| EBVNW | 0.028 | 0.021 | 0.17 |
| EBVLW | 0.81 | 0.74 | 0.27 |
| EBVDAYS | 0.48 | 0.96 | 0.62 |
| EBVBF | −0.006 | 0.017 | 0.71 |
| EBVLEA | −0.016 | 0.038 | 0.68 |
| EBVADG | −0.01 | 0.021 | 0.64 |
| EBVFG | 0.0031 | 0.016 | 0.84 |
| EBVADF | −0.01 | 0.017 | 0.58 |

TABLE 29
Dominance effects of B allele for FS1 marker Landrace

| Trait | Effect | Standard Error | P value |
|---|---|---|---|
| EBVLS | −0.12 | 0.14 | 0.43 |
| EBVNW | 0.02 | 0.03 | 0.49 |
| EBVLW | 0.18 | 1.1 | 0.87 |
| EBVDAYS | −1.5 | 1.8 | 0.42 |
| EBVBF | 0.02 | 0.04 | 0.72 |
| EBVLEA | 0.02 | 0.08 | 0.78 |
| EBVADG | 0.03 | 0.04 | 0.43 |
| EBVFG | −0.01 | 0.03 | 0.67 |
| EBVADF | 0.03 | 0.04 | 0.52 |

Least square (LS) means were calculated for each trait and are presented in Tables 30 and 31. When looking at pair wise differences of LS means for each genotype in the Yorkshire group when considering litter size EBV, BB differed significantly from AB and AA (P=0.015, 0.015, respectively). When examining number weaned EBV AA differed significantly from AB (P=0.05). When examining litter weight EBV AB tended to differ significantly from AA (P=0.097). In the Landrace group, the only pairwise difference in LS means that tended towards significance was for number weaned EBV for BB versus AA (P=0.1).

TABLE 30
a. Least Square Means for Reproductive Trait Estimated Breeding Values
for Yorkshire

| | LS | SE | NW | SE | LW | SE |
|---|---|---|---|---|---|---|
| AA | −.01 | .03 | −.02 | .01 | −.36 | .37 |
| AB | .12 | .04 | .02 | .02 | .82 | .6 |
| BB | .13 | .06 | 0 | .02 | .37 | .8 |

b. Least Square Means for Growth Trait Estimated Breeding Values for
Yorkshire

| | DAYS | SE | BF | SE | LEA | SE | ADG | SE | FG | SE | ADF | SE |
|---|---|---|---|---|---|---|---|---|---|---|---|---|
| AA | .09 | .48 | .01 | .01 | −.05 | .02 | 0 | .01 | .01 | .01 | .01 | .01 |
| AB | .29 | .77 | 0 | .01 | −.08 | .03 | −.01 | .02 | 0 | .01 | −.01 | .01 |
| BB | −.45 | 1 | 0 | .02 | −.07 | .04 | .01 | .02 | −.01 | .02 | 0 | .02 |

TABLE 31
a. Least Square Means for Reproductive Trait Estimated Breeding Values
for Landrace

| | LS | SE | NW | SE | LW | SE |
|---|---|---|---|---|---|---|
| AA | .07 | .06 | .03 | .01 | .85 | .44 |
| AB | −.1 | .07 | .01 | .02 | .08 | .49 |
| BB | .01 | .25 | −.06 | .06 | −1 | 1.8 |

b. Least Square Means for Growth Trait Estimated Breeding Values for
Landrace

| | DAYS | SE | BF | SE | LEA | SE | ADG | SE | FG | SE | ADF | SE |
|---|---|---|---|---|---|---|---|---|---|---|---|---|
| AA | −.14 | .77 | −.01 | .02 | 0 | .03 | 0 | .02 | −.01 | .01 | 0 | .02 |
| AB | −.77 | .85 | 0 | 0.2 | −.01 | .04 | .02 | .02 | −.01 | .01 | .01 | .02 |
| BB | 1.7 | 3.2 | −.02 | .07 | −.07 | .14 | −.03 | .07 | .01 | .05 | −.04 | .07 |

Discussion and Conclusions: The data from these two small groups provided some useful information. In the case of NSB in the Yorkshire groups, the negative additive effect of the B allele tended towards significance. The trends were in the favorable direction for all additive effects when examining phenotypic data in both populations. This also held true for the LS means of the raw data of both breeds. A significant positive additive effect was found in the Yorkshire group for EBV of litter size. The EBVs in the Yorkshire groups were in the favorable direction for number weaned, litter weight, days to reach 250 lbs., backfat, average daily gain and feed to gain ratio. The Landrace EBV results are not as promising, with a somewhat significant negative additive effect on NW and the only additive effects that were in the favorable direction for the given trait were backfat and average daily gain. The LS Means of EBVs for the Yorkshire group were in the favorable direction for litter size, days to reach 250 lbs., backfat, average daily gain and feed to gain ratio. The LS Means for the Landrace population were not in the favorable direction for EBVs. These results are not a replica of examples 1 and 2, but they do show that the follistatin markers may be useful in selecting for growth and reproductive traits in breeds other than Chester Whites. The patterns may very well have been the same had a larger group of each breed been tested.

### EXAMPLE 6

Statistical Analysis of all Data Compiled: Statistical calculations were performed on the entire data set, using all individuals from examples 1-5, for FS1 marker. Data were analyzed using PROC GLM (SAS Inst., Inc., Cary, N.C.). A total of 1464 litter records and 730 individuals were used. Because this data set included different breeds and populations, the statistical model was altered in order to account for other sources of variation. When examining phenotypic data, the model included fixed effects for genotype, breed, farm, parity and farrowing season. Farrowing season, farm and parity proved to be significant sources of variation for all independent variables examined. Breed was a significant source in several traits as well. Even with all these other sources of variation, genotype was found to be a significant, contributing factor to the differences seen among individuals for the traits of NBA, NSB, average weaning weight and number after transfer (P=0.0032, P=0.0797, P=0.0254, P=0.0426, respectively). Average weaning weight (AWW) is the average weaning weight of an individual pig. Because on commercial farms, efforts are made to prevent sows from having to nurse too many piglets by cross fostering, number after transfer was a trait examined as well.

Additive (a) and dominance (d) effects were calculated using estimate statements with orthogonal contrasts of solutions for genotypic effects. Additive effect is defined as half the difference between LS means of the two homozygotes. Dominance effect is the heterozygote LS mean minus the average of the two homozygote LS means. Additive and dominance effects are listed in Tables 32 and 33.

TABLE 32
Additive effects of B allele for FS1 marker

| Trait | Effect | Standard Error | P value |
|---|---|---|---|
| # Born Alive | 0.45 | 0.25 | 0.08 |
| Birth Weight | 0.57 | 0.84 | 0.5 |
| # Stillborn | −0.29 | 0.16 | 0.07 |
| Mummies | −0.09 | 0.08 | 0.25 |
| Weaning Weight | 6.2 | 3.5 | 0.08 |
| # Weaned | 0.35 | 0.25 | 0.17 |
| Avg. Weaning Wt. | 0.77 | 0.4 | 0.05 |
| # After Transfer | 0.54 | 0.28 | 0.06 |

TABLE 33
Dominance effects of B allele for FS1 marker

| Trait | Effect | Standard Error | P value |
|---|---|---|---|
| # Born Alive | −0.48 | 0.16 | 0.003 |
| Birth Weight | −1.06 | 0.53 | 0.04 |
| # Stillborn | −0.17 | 0.1 | 0.09 |
| Mummies | −0.03 | 0.05 | 0.49 |
| Weaning Weight | 0.17 | 2.2 | 0.94 |
| # Weaned | −0.08 | 0.16 | 0.63 |
| Avg. Weaning Wt. | 0.33 | 0.25 | 0.19 |
| # After Transfer | −0.38 | 0.18 | 0.03 |

Least square (LS) means were calculated for each trait and are presented in Table 34. When looking at pair wise differences of LS means for each genotype the genotypes were found to differ for several traits. For the trait NBA, BB tended to differ from AA (P=0.08) and differed significantly from AB (P=0.004). For BW trait AB tended to differ from BB (P=0.09). When considering the number of stillborns AA differed from both BB and AB (P=0.0014, 0.0677, respectively). For WW trait, AA differed from AB (P=0.079). Genotype AA differed significantly from both AB and BB when looking at AWW (P=0.004, 0.05, respectively). Individuals with the BB genotype differed from AA and AB individuals when considering the NAT (P=0.056, 0.015, respectively).

TABLE 34
Least Square Means for Litter Traits and Standard Errors

| | NBA | SE | BW | SE | NSB | SE | MUM | SE | WW | SE | NW | SE | AWW | SE | NAT | SE |
|---|---|---|---|---|---|---|---|---|---|---|---|---|---|---|---|---|
| AA | 9.8 | .3 | 27 | 1 | 1.1 | .2 | .17 | .1 | 99.7 | 4 | 7.8 | .3 | 11.7 | .5 | 9.6 | .3 |
| AB | 9.5 | .3 | 26 | 1 | .8 | .2 | .09 | .1 | 103 | 4 | 7.9 | .3 | 12.4 | .5 | 9.5 | .3 |
| BB | 10.2 | .4 | 28 | 1.3 | .8 | .2 | .08 | .1 | 106 | 5 | 8.1 | .4 | 12.5 | .6 | 10.1 | .4 |

The same statistics were also run using estimated breeding values for the following traits: litter size (EBVLS), number weaned (EBVNW), litter weight (EBVLW), days to 250 lbs. (EBVDAYS), backfat (EBVBF), loin eye area (EBVLEA), average daily gain (EBVADG), feed to gain ratio (EBVFG), average depth of fat (EBVADF). Fixed effects in this model included genotype, breed and farm. Genotype was found to be significant when considering the breeding value for litter size (P=0.0399) and number weaned (P=0.0115). Additive and dominance effects are summarized in tables 35 and 36.

TABLE 35
Additive effects of B allele for FS1 marker

| Trait | Effect | Standard Error | P value |
|---|---|---|---|
| EBVLS | 0.03 | 0.04 | 0.47 |
| EBVNW | 0.02 | 0.01 | 0.035 |
| EBVLW | 0.74 | 0.42 | 0.077 |
| EBVDAYS | −0.8 | 0.48 | 0.096 |
| EBVBF | −0.01 | 0.01 | 0.28 |
| EBVLEA | −0.03 | 0.02 | 0.2 |
| EBVADG | 0.02 | 0.01 | 0.076 |
| EBVFG | −0.02 | 0.01 | 0.04 |
| EBVADF | 0.03 | 0.04 | 0.47 |

TABLE 36
Dominance effects of B allele for FS1 marker

| Trait | Effect | Standard Error | P value |
|---|---|---|---|
| EBVLS | −0.06 | 0.03 | 0.048 |
| EBVNW | 0.009 | 0.008 | 0.25 |
| EBVLW | −0.09 | 0.28 | 0.76 |
| EBVDAYS | 0.43 | 0.32 | 0.18 |
| EBVBF | −0.002 | 0.007 | 0.78 |
| EBVLEA | 0.03 | 0.01 | 0.05 |
| EBVADG | −0.01 | 0.007 | 0.17 |
| EBVFG | 0.005 | 0.006 | 0.37 |
| EBVADF | −0.005 | 0.006 | 0.4 |

Least square (LS) means were calculated for each trait and are presented in Table 37. When looking at pair wise differences of LS means for each genotype for EBVLS BB tended to differ from AB (P=0.084). For the trait EBVNW, AA individuals differed from both AB and BB individuals (P=0.0066, 0.035, respectively). When looking at EBVLW, BB tended to differ from AA (P=0.077). For the EBV for days to reach 250 lbs., BB tended to differ from both AA and AB (P=0.096, 0.075, respectively). When examining EBV for loin eye area BB differed from AB (P=0.044). Individuals with the BB genotype differed from AA and AB for EBVADG (P=0.076, 0.063, respectively). The BB individuals also differed from both AA and AB for EBVFG (P=0.041, 0.096, respectively).

TABLE 37
a. Least Square Means for Reproductive Trait Estimated Breeding Values

| | LS | SE | NW | SE | LW | SE |
|---|---|---|---|---|---|---|
| AA | .06 | .03 | 0 | .01 | −.01 | .3 |
| AB | .02 | .03 | .02 | .01 | .27 | .3 |
| BB | .09 | .05 | .02 | .01 | .73 | .5 |

b. Least Square Means for Growth Trait Estimated Breeding Values

| | DAYS | SE | BF | SE | LEA | SE | ADG | SE | FG | SE | ADF | SE |
|---|---|---|---|---|---|---|---|---|---|---|---|---|
| AA | −.66 | .4 | .01 | .01 | −.03 | .02 | .01 | .01 | 0 | .01 | .01 | .01 |
| AB | −.62 | .4 | 0 | .01 | −.02 | .02 | .01 | .01 | −.01 | .01 | .01 | .01 |
| BB | −1.5 | .5 | 0 | .01 | −.06 | .02 | .03 | .01 | −.02 | .01 | .01 | .01 |

Conclusions and Discussion: Genotype was found to be a significant source of variation for several traits. Based on this information it can be concluded that the FS1 marker can be used to explain some of the variation among individuals when considering phenotypic litter data and breeding values as well as growth trait EBVs.

Significant or somewhat significant additive effects were found for several phenotypic litter traits, including NBA, NSB, WW, AWW, and NAT. There was a positive additive effect associated with the B allele for NBA, WW, AWW, and NAT. This means that for every additional B allele an individual has there is an increase in that particular trait. For example, with NBA, the additive effect was 0.45, so for every additional B allele, you can expect NBA to increase by 0.45 pigs per litter. Likewise with a negative additive effect, as in the case of NSB, there is a decrease in NSB associated with each additional B allele. Significant dominance effects indicate that heterozygotes have an advantage or disadvantage over the homozygotes. In the case of NSB, the heterozygous individuals have fewer stillborns, than the average of the opposite homozygotes. Even when the additive effects weren't found to be statistically significant, they were in the favorable direction for each phenotypic litter trait analyzed.

When examining the LS means for raw litter data, BB individuals were found to be superior to other genotypes for the traits NBA, MUM, WW, NW, AWW, and NAT. These individuals averaged 0.55 more pigs born alive, 0.05 fewer mummified fetuses, 4.65 pounds heavier per litter at weaning, 0.25 more pigs weaned per litter, 0.45 pounds heavier per individual weaned, and 0.55 more pigs after transfer than the average of the alternative genotypic groups.

Additive and dominance effects for breeding value data were found to be significant for some of the traits. Significant favorable additive effects were found for EBVNW, EBVLW, EBVDAYS, EBVADG, and EBVFG. Favorable dominance effects were seen for EBVLEA. Even though the additive effects were not significant for EBVLS and EBVBF, they were in the favorable direction.

As with LS means of raw litter data, BB individuals were found to be better than the other groups in several EBV categories. This was the case with the following traits: EBVLS, EBVNW, EBVLW, EBVDAYS, EBVBF, EBVADG, and EBVFG. Because the marker appears to account for variation among individuals when examining breeding values, it is a more firm indication that the marker can be used to make selection decisions for reproductive traits. This is because breeding values are a more accurate indicator of an individual's genetic ability, than just raw phenotypic data. This is due to the fact that breeding values are calculated using data from an individual's relatives, as well as its own data, to determine the breeding value of a particular trait. It is also apparent that the marker can be used for making not just reproductive selection decisions, but growth performance selections as well.

### EXAMPLE 7

Sequencing PCR Products:

The PCR products were sequenced.

### Equivalents

Persons skilled in the art will identify, or be able to determine through routine experiment, other assays and methods to test for the follistatin genetic markers described in this disclosure. Such other methods are intended to be encompassed by this disclosure.

What is claimed is:

1. Genetic mutations within the porcine follistatin gene which have been found to be associated with overall reproductive performance and growth traits in pigs.

2. A method of screening pigs to determine those more likely to produce larger litters, possess other desirable reproductive traits and grow more efficiently and produce offspring also possessing those same abilities, which is comprised of: obtaining a sample of genetic material from a pig and analyzing the genomic material obtained in (i) to determine which follistatin (FS) allele(s) is/are present in said sample.

3. A method as claimed in claim 2 wherein the determination of FS alleles in the analysis step encompasses determining the presence of at least one allele associated with at least one polymorphism in FS.

4. A method as claimed in claim 3 wherein the DNA markers are single nucleotide polymorphisms.

5. A method as claimed in claim 4 wherein the DNA marker is FS1 or FS2.

6. The method of claim 2 wherein said method of identifying the presence or absence of a polymorphism is selected from a group consisting of: restriction fragment length polymorphism (RFLP) analysis, heteroduplex analysis, single strand conformational polymorphism (SSCP), denaturing gradient gel electrophoresis (DGGE), temperature gradient gel electrophoresis (TGGE), DNA sequencing or other similar technique that can be utilized to detect differences in sequence between DNA strands.

7. The preferred method of claim 2 wherein said step of assaying for the presence of said polymorphism comprises the steps of: digesting said genetic material with a restriction enzyme that cleaves the pig follistatin gene in at least one place, separating the fragments obtained from said digestion, detecting a restriction pattern generated by said fragments, and comparing said pattern with a second restriction pattern for the pig follistatin gene obtained by using said restriction enzyme, wherein said second restriction pattern is associated with increased litter size, number born alive, number weaned, weaning weight, average daily gain, and feed to gain conversion as well as a decreased number of stillborns, and days to reach 250 lbs.

8. The method of claim 7 wherein said restriction enzyme is Mspl or other such restriction endonuclease that will differentially cut at FS1 polymorphism.

9. The method of claim 7 wherein said restriction enzyme is Fnu4Hl or other such restriction endonuclease that will differentially cut at FS2 polymorphism.

10. The method of claim 7 wherein said separation is by gel electrophoresis.

11. The method of claim 7 wherein said step of comparing said restriction patterns comprises identifying specific fragments by size and comparing the sizes of said fragments.

12. The method of claim 7 further comprising the step of amplifying the pig follistatin gene or a portion thereof which contains said polymorphism, prior to said digestion step.

13. The method of claim 7 wherein said polymorphism is a polymorphic Mspl restriction site if said amplification is of the region including exons 2 and 3 as well as the sequence between in the pig follistatin gene.

14. The method of claim 7 wherein said polymorphism is a polymorphic Fnu4Hl restriction site if said amplification is of the region including exons 3 and 4 as well as the sequence between in the pig follistatin gene.

15. The method of claim 12 wherein said pig follistatin gene is located on chromosome 16.

16. The method of claim 7 wherein said amplification is of the sequence spanning from exon 2 thru exon 3 or the sequence from exon 3 thru exon 4 includes selecting a forward and reverse sequence primer capable of amplifying a region pig follistatin gene which contains a polymorphic Mspl site or Fnu4Hl site.

17. The method of claim 16 wherein said forward and reverse primer sets amplify the region on chromosome 16 associated with the pig follistatin gene.

18. The method of claim 16 wherein said forward and reverse primers amplify a polymorphism found in the sequence of the follistatin gene, especially if that region covers the sequence from exon 2 thru exon 3 or exon 3 thru exon 4.

19. The method of claim 16 wherein said primers are SEQ ID NO: 1 and SEQ ID NO: 2 for FS1 marker and SEQ ID NO: 3 and SEQ ID NO: 4 for FS2 marker.

20. The method for identifying a polymorphism for pig reproductive and growth traits comprising the steps of: determining the reproductive and growth traits of each animal, determining the polymorphism in the follistatin gene of each pig wherein the polymorphism is identifiable by amplification by a set of primers selected from the group consisting of the set of a forward primer SEQ ID NO: 1 and reverse primer SEQ ID NO: 2 or the set of a forward primer SEQ ID NO: 3 and reverse primer SEQ ID NO: 4, and associating the reproductive and performance traits of each pig with said polymorphism thereby identifying a polymorphism for pig reproductive and growth traits.

21. The method of claim 20 further comprising selecting pigs for breeding which are predicted to have favorable reproductive and growth traits by said marker.

22. The method of claim 20 wherein said analysis comprises digestion of PCR amplified DNA with the restriction enzyme Mspl if said amplification is of the region from exon 2 thru exon 3 or Fnu4Hl if said amplification is from exon 3 thru exon 4.

23. The method of claim 20 wherein said polymorphism associated with favorable reproductive and growth traits is amplified by use of forward and reverse primers comprising at least 4 consecutive bases in SEQ ID NOS: 1 and 2 or 3 and 4.

24. The method for determining the presence of a polymorphic site in the follistatin gene which is associated with favorable reproductive and growth traits in pigs comprising: obtaining genetic samples from male and female pigs of the same breed, or breed cross, or derived from similar genetic lineages grown under normal conditions, determining reproductive traits of each pig from which a genetic sample was obtained, analyzing the genetic samples for polymorphisms in a gene associated with favorable reproductive and growth characteristics wherein the gene is the follistatin gene wherein the polymorphisms are identifiable by amplification by a set of primers selected from the group consisting of the set of a forward primer SEQ ID NO: 1 and reverse primer SEQ ID NO: 2 or the set of a forward primer SEQ ID NO: 3 and reverse primer SEQ ID NO: 4 or a comparable set of primers, performing an appropriate restriction digest or similar analysis to determine which FS alleles are present, and correlating the polymorphism(s) to reproductive and growth traits by comparing the presence of polymorphisms to each animals' records.


SEQUENCE LISTING (7 sequences)

SEQ ID NO: 1 - DNA, 20 bp, Sus scrofa
ggaccgaggaggacgtaaat

SEQ ID NO: 2 - DNA, 20 bp, Sus scrofa
ggcctttccaggtgatgtta

SEQ ID NO: 3 - DNA, 20 bp, Sus scrofa
tgccgaatgaacaagaagaa

SEQ ID NO: 4 - DNA, 20 bp, Sus scrofa
cagaaaacatcccgacaggt

SEQ ID NO: 5 - DNA, 631 bp, Sus scrofa
ggaccgaggaggacgtaaatgacaacacacttttcaagtggatgattttcaatggaggtgcccccaactgcatcccatgtaaaggtaagggtcttcctccccaatttgcaggccctcagtagaggggtcttaccctcagcttccccactgcccagctggcgtttgtttgagcctgggagagctttgttggtgggttccccctaactgcctccagccccagtaagccattgggcgttgaattttggcagtcgtataccacaggcaaaacaagatctgcagaggttcgctgacctcttagccaaatgcggtggtgacaccagccagccctctcactttgagagagatttgggtatgggaacacaaatctgtcccccacccctcctcaatttctgggcgagagcctagacccctcaggcttgaactcccggctcgcgattgcgcaaggcacccgaagccctcctggctgacctgtttggtgcctggccctggttttaatcccctgcctctttccaactcttagaaacgtgcgagaacgtggactgtgggcccgggaaaaaatgccgaatgaacaagaagaacaaaccccgctgcgtctgcgccccggattgttctaacatcacctggaaaggcc

SEQ ID NO: 6 - DNA, 504 bp, Sus scrofa
tgccgaatgaacaagaagaacaaaccccgctgcgtctgcgccccggattgttctaacatcacctggaaaggcccagtctgtgggctggatgggaaaacctaccgcaacgaatgtgctctcctcaaggccagatgtaaagagcagccggaactggaagtccagtaccaaggcaaatgtaaaagtaggtcacccctcctccagctttcaacctgaggtagtcccgcagcaagacccctggggtttggtgtagtcacagtaagagcctaatgatatcaaataaaggaacccttttctaatgactccttaagaacgctgaaggcaacccagaggcacagggtttttttttttgaaaaccacagcgttcccaaaataaatttttgaaagctggatgcttccattcatgatttccttgatagtatcaaatgctgagtcccaaagtacaggaaggtgcctattaacgtgtgtttctctctttgtttcagagacctgtcgggatgttttctg

SEQ ID NO: 7 - DNA, 6247 bp, Sus scrofa
tgtcccgcgctaggttacaggggcctaaacaccagcgacagacccggagtggggtgtttcagcagctcggggatcgccgggcatttgcaaaagtcttcttggagcggagcggcgatgcgaagaccaattccgcccgctctcgtccgccagccgccacccccagcccaccatgccccccccaacgcacacactcacacaccaccatgaacgaacacacacacacacacacacacacacgccgtcccaggcgcgccggcgctgcctgcgagcggcggcgtgcaggacttgaagtgggtgttcttccccactccccacccccgaagcgtcgccctcacccccctccgtcccggtcgcctgccccctccctctcgctcctacgcaaataagaactcggcgataccctcctgccgctttgatttcgggcacctccaccagataattgggaagggtttccagaaggtgggaaatgtcacctgattcacactgaacttttaaaagctccccacccccaaggagccgcgcacaccctcgctcgaggccgccctcccactgccccacacactgggagaccgcccaccgcaaaccgcggagacccccgtctagatttaaagcgcggctgcgcccggcttctgacgtccattgaatcgcgcgggcggccggcggcgagcgcggggttgcgccgggatcgctgcgccctccgccgctcgcctctgcgacgcgcgccgctcgccggaaccactcgccgccgcgcctgctccccgcaccgtcgcgctcctcgtcccgcgcctgaccccagaatggtccgtcccaagcaccagcccggcgggctatgcctcctgctgctgctgctctgccagttcatggaggaccgcagcgcccagggtaagagaagggggatgcgctgggaggctcggcaggcgacaggggtttgtacttttcagtcgtctccactggtctgttctgagccctgctcttagatgttggaccaacttaggaagtccggatggagaggagtgggaaaattgaaggcagtgacactttaggactgggagccgagtttggagcggggcgatcctttaaaaggaaagggagtgagctggagtctgtctcagtagttagggttagatgctccaagttctggacataatgagtttagatgttatctcccctgtttggctctgaagaaggggaaaagcatcagtagcctggagatttctgtcgacccacttacagtctacctctctacctcctacatcttttggatactttaaaaaatgactgcattggggagggggatactgaaatttcttcggtggctgagggacgtggtttcagaaactttgcccaaacttggaaatgtgggtgccgaaaattttagagggaagtagctttctgcctagtggatgcttcttccaacaccctcccccaaacccgcaaaaaaaaaaaaaaaaaaaaaaaaatacccctgactgttttcagtagtttacaattttttttctttgcctcattcctcatgttaactttctgtagaacagtgaaaagcccacaattgatttaaattctgagatgcagtaagagcaaagtatttttttaattagatttatttatttctgctagtctttgggcagacttttttaaaatcataaaagcagcttgctgttcaatatattgttaagattatagggatgattcccgagaggtctttctgaccaaatccgaacattaaaaaggaggagggggggggaattacttgcaaagatttcttggtaggtatgagatatgtattaccatgagacggtggaagcccctcccccctttattattattatttacttggttggttgggttttttccccctacactcttcttgagacggacttgtaactttcctggctttagcattaacattttatgtgtgttgtaaactggccaccgggcactgcccctcgcggtctgcaacgcaccactctgagttgcccgcctttcggggcgaggcgctgcctaggtgccggggacgtgggcgccggggaaagcagccctcggagatttcgaaagatcgcggtggccgcgggcgtcctctggatctggcggattgcaaaacgggaccgtcgggagggctgcgcttgaggccgcacggggcagctccgggcaaggctcagttgcttttgcttttttgctttcatttttctcaagtgccttttgcttgatctgttcctgtaggtgtctgtctcagcctctccaccctcccccccttttttctttctcttaagttctcctactttcttaacccctgtcacaaataatacttttaccgcccacatgaaaccaacatgtaaaaacatccgtcgcatcctgtttttgtcaggttctttaatctgctcttcgagtcgctgcaggttatgaaataggacgaataaaagtaaacagtctagtaaaagtcaatgcaagctgcacgtgttgtgtctgggtcactggtaactgacattgatatggctggggcgctctggcttctcgctctcgccctccctccccaccgccccctcgcccacctcccacttcttcaatctggctccccccctttacacaccccgtcattcccacctatttattttctgcttttctcttctctctcgcctcctcctccacgctcactccctccccatccccgaagggtttccttgcctcgaaagctcaccgcctccctacccgaacccctccgctcacttgcccacctccccacccctgtctccccgcagctgggaattgctggctccgccaagcaaaaaacggccgctgccaggtcctgtataaaaccgaactgagcaaggaggagtgctgcagcaccggccgcctgagcacctcctggaccgaggaggacgtaaatgacaacacacttttcaagtggatgattttcaatggaggtgcccccaactgcatcccatgtaaaggtaagggtcttcctccccaatttgcaggccctcagtagaggggtcttaccctcagcttccccactgcccagctggcgtttgtttgagcctgggagagctttgttggtgggttccccctaactgcctccagccccagtaagccattgggcgttgaattttggcagtcgtataccacaggcaaaacaagatctgcagaggttcgctgacctcttagccaaatgcggtggtgacaccagccagccctctcactttgagagagatttgggtatgggaacacaaatctgtcccccacccctcctcaatttctgggcgagagcctagacccctcaggcttgaactcccggctcgcgattgcgcaaggcacccgaagccctcctggctgacctgtttggtgcctggccctggttttaatcccctgcctctttccaactcttagaaacgtgcgagaacgtggactgtgggcccgggaaaaaatgccgaatgaacaagaagaacaaaccccgctgcgtctgcgccccggattgttctaacatcacctggaaaggcccagtctgtgggctggatgggaaaacctaccgcaacgaatgtgctctcctcaaggccagatgtaaagagcagccggaactggaagtccagtaccaaggcaaatgtaaaagtaggtcacccctcctccagctttcaacctgaggtagtcccgcagcaagacccctggggtttggtgtagtcacagtaagagcctaatgatatcaaataaaggaacccttttctaatgactccttaagaacgctgaaggcaacccagaggcacagggtttttttttttgaaaaccacagcgttcccaaaataaatttttgaaagctggatgcttccattcatgatttccttgatagtatcaaatgctgagtcccaaagtacaggaaggtgcctattaacgtgtgtttctctctttgtttcagagacctgtcgggatgttttctgtccaggcagctccacatgtgtggtggaccagactaataatgcctactgtgtgacatgtaaccgcatttgcccagagcccacctcctcagaacagtatctctgtgggaatgatggagtgacctactccagtgcctgtcacctgagaaaggctacctgcctactgggcagatctattggattggcctatgagggaaagtgtatcagtaggtattctggattgagaaaggaaaagaaataaatgggaaaagggaaaaaggctaattctgtcattaaaaaaagcctaaagcctccccaaacacatagcttcagcaaagggagaaatattctccagtttgggtaaactgtcctgctcacagcactgtcttccagaagcatcagcacatatattgaaatggcagcaagacacaggaaacaattttccttcatagaaacccaagacctcactcacttttacacatcttttaaagtgctagacttgctggaagcaagaaataattacttaacagttcctaaaatctgttctcagattctgataactaatggaacttcttttttttaagatcccacttatgggaaataaataaatatgtatatttaactcacagatattgtgaaatcctgttcccccaaaaaaactacagtattttcatgcactaattatagtatgaagggtgtgtgtgtgtgtgttgagtttcaccggttttttaaaagaacatttttgcaccatatctccattaacccccttaatgagtaataaggtatgttatatgtttacatttattgataggagactatagagaaaagggggcaagggggaaatggatttactcatcacaggtgtattatatcctagaagcaaagtcctgtgaagacatccagtgcactggtggaaaaaagtgcttatgggatttcaaggttggcagaggccgctgttccctctgcgatgagctgtgccctgagagtaagtctgaggagcctgtctgtgccagtgacaatgccacctacgccagtgagtgtgccatgaaggaagcggcctgttcctcaggtgtgctgctggaagtaaagcactctggatcttgcaactgtaagtgtgatttctaacctttctgaaattgaaagctttcccaggcaggccctagggaatgcgatgtaaaaaagcacaaaaacctccccatataagcccagttaggtagctgctaagttttaccagcaattcagcaatccacagaaaaactgcctggaatttttcctgggcccttaggacttgtcaacgatcatcaatggggtcgcctctataaacctgtttccagtcattcacccctaatttctttagctttagtagtgtgctttgctttattaacacttgaatctaaactaactgcttcaaaggcgtttttttctttttcaaagacagctttctattatcatacatgggctgctgctttgcgcagttgcctctactaactctgaattaagaacccaaagcagttatacctagaacacgagcgctttttatctaatttcaggaatctgcccgtaaaacctgagccattgattcttcagaactttctgcagtttttgacttcatagataatgtttttaaaaaacattttttttaaacttaatgcataacagcagatgccaaaaagcaaaaaaaaaaccaaaaaaaaaaaaaagcatcttactgcaagtcacataaaaatgcaacgctgtaatatggctatttcagagggctttgaaaacatacactgagctgcttctgcgctgttgttgtccgtatttaaacagcagctcccctgtattcccccatctagccatttcagaagacaccgaggaggaggaggaagatgaagaccaggactacagctttcctatatcttctattctagagtggtaaaccctccaccaattttcagtgttgacatagcctttgggcaaaaaaaaaaaaaaaaaaaaaaaaagaaagaaaaaagaaaaaaaaagaaagaaagaaagagaaaaaagaaaaatatatattgtccatactgtaaataagtgtatgcttatttatttggggggaaaagtatacataaaggacctttgtcctaaagctctctcccaggccaccttgttactcattggacccggagaggcagtcattgtgaggtctactggatgaggcccatagttgagacttgtagacatttatttatactgtgtcatgttttataatttatacataaaatgtctggttgactgtacaccttgtttttgaagaaatttattcgtgaaaggaagagcagttgttatttattgtgaggtctcttgcttgtaaagtaaaa